(19) United States
(12) Patent Application Publication
(10) Pub. No.: US 2022/0296949 A1
(43) Pub. Date: Sep. 22, 2022

(54) CARBON-BASED FILTERS FOR USE IN ELIMINATING PATHOGENS

(71) Applicant: University Of Cincinnati, Cincinnati, OH (US)

(72) Inventors: Vesselin N. Shanov, Cincinnati, OH (US); Soryong Chae, Cincinnati, OH (US); Yanbo Fang, Cincinnati, OH (US); Hyunsik Kim, Cincinnati, OH (US); Yoontaek Oh, Cincinnati, OH (US); Hung Nguyen, Cincinnati, OH (US)

(21) Appl. No.: 17/655,688

(22) Filed: Mar. 21, 2022

Related U.S. Application Data
(60) Provisional application No. 63/196,155, filed on Jun. 2, 2021.
(60) Provisional application No. 63/163,163, filed on Mar. 19, 2021.

(51) Int. Cl. A62D 9/00; A61L 9/00

(52) U.S. Cl. CPC A62D 9/00; A61L 9/00; A61L 2209/21; A61L 2209/16

(57) ABSTRACT

The present invention relates to a carbon-based material for use in a piece of personal protective equipment and an air filtration system. The carbon-based material may function as a filter by providing a tortuous path for a pathogen to traverse. The carbon-based material may be used as a carbon-based heater that can reach a pathogen inactivation threshold temperature to enable heat inactivation of one or more pathogens. In embodiments where a piece of personal protective equipment includes a carbon-based heater, an insulating layer may be included to attenuate the temperature generated by the carbon-based heater from the face of a user.

### CROSS-REFERENCE TO RELATED APPLICATIONS

This application claims the benefit of the filing date of U.S. Provisional Application No. 63/163,163, filed on Mar. 19, 2021, and U.S. Provisional Application No. 63/196,155, filed on Jun. 2, 2021, the disclosures of which are incorporated herein by reference in their entireties.

### STATEMENT REGARDING FEDERALLY FUNDED RESEARCH DEVELOPMENT

This invention was made with government support under grant number T420H008432 awarded by the Centers for Disease Control and Prevention (CDC) and Grant No. 2028625 awarded by the National Science Foundation. The U.S. Government has certain rights in the invention.

### FIELD OF THE INVENTION

The present invention generally relates to apparatus for the filtration of pathogens. More specifically, the invention relates to apparatus incorporating a carbon-based heating element to facilitate filtration and elimination of pathogens.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

First responders, especially doctors and nurses, are constantly exposed to harmful pathogens, including biological exposure to tuberculosis bacteria (Mycobacterium tuberculosis), human immunodeficiency virus (HIV), Ebola virus, Coronavirus, etc. For at least this reason, Personal Protective Equipment (PPE), such as facemasks and respirators, is imperative for the safety of healthcare workers to mitigate the variety of infectious risks. Unfortunately, PPE remains in short supply worldwide. Further, conventional facemasks and respirators, because of their single functional feature of simple filtration, cannot completely trap all biological hazards. And, since conventional masks only contain a simple filter, they lack the ability to positively inactivate pathogens trapped in the mask (i.e., an inactivation that is not simply the natural expiration of the pathogen while, or due to being, trapped). These failures significantly increase the risk of infection and impairs the facemask's ability to protect healthcare workers. With approximately 59 million healthcare workers worldwide, even a 0.1% PPE malfunction rate could lead to tens of thousands of healthcare workers being directly exposed to hazards.

Moreover, since pathogens may remain airborne for hours, another method of reducing pathogens is to eliminate them from the air in a given space. Conventional methods for removing pathogens from the air in a given space include heating, ventilation, and air conditioning (HVAC) systems, high efficiency particulate air (HEPA) filters, and other similar systems for filtering particulate matter. These systems are employed in a variety of contexts including homes, offices, vehicles, and other enclosed spaces. However, since these systems conventionally only include a filter for trapping particulate matter, they also lack the ability to positively inactivate pathogens. These failures allow the pathogens not trapped by the systems to continue to pose a risk to those exposed.

The integration of a heater into the facemask or system for filtering particulate matters enables a dual functionality. One function may be to increase the ability of the facemask or system to trap pathogens. Another function is to enable the facemask or system to eliminate pathogens via thermal treatment. To date, methods of providing a heat source in a facemask or filter include use of a conventional metal-based heater. However, implementation of a metal-based heater into a conventional facemask faces several hurdles. First, conventional metal-based heaters add significant weight to the facemask or filter. Second, many metal-based heaters do not significantly increase the ability of the facemask to filter pathogens. Third, metal-based heaters can be costly to implement compared to alternatives.

Accordingly, there is a need for PPE (such as a facemask) and equipment (such as an air filtration system) that has an enhanced ability to trap and/or eliminate pathogens.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

One aspect of the present invention is directed to an apparatus that provides enhanced filtration of pathogens and/or other methods of trapping, reducing, or eliminating pathogens—and thus provide enhanced protection to an individual. One embodiment of such an apparatus may include a piece of personal protective equipment and a carbon-based material. The piece of personal protective equipment may be a mask, such as one that may be typically worn to prevent pathogens from entering a body via the nose or mouth. Certain such masks include an inner layer and an outer layer, and in various embodiments of the present invention, the carbon-based material may be disposed relative to the mask at a location selected from an external surface of the inner layer, an external surface of the outer layer, and between the inner and outer layers. In some embodiments, the carbon-based material may include a carbon veil. In some embodiments, the carbon-based material includes a carbon nanotube sheet. The presence of the carbon-based material may provide a filtration function to the apparatus (or may provide an additional or enhanced filtration function to a level of filtration that is already provided by the layer or layers of the mask itself).

Another aspect of the present invention is directed to an apparatus that includes a heating function to reduce or eliminate pathogens—and thus provide enhanced protection to an individual. Some embodiments in accordance with this aspect of the invention may include the apparatus (i.e., personal protective equipment with carbon-based material) described above. In certain embodiments, the apparatus further includes a plurality of electrodes, a plurality of wires, a power source, and a switch, wherein the plurality of electrodes, plurality of wires, power source, and switch are operatively connected to the carbon-based material, to form a carbon-based heater. The carbon-based heater can reach a pathogen inactivation threshold temperature when a voltage is applied to the carbon-based material.

Another aspect of the present invention is directed to an air filtration system that—like the apparatus described above—may include a carbon-based material that can provide a filtration function and/or a heating function. Certain embodiments of the air filtration system include an air flow path that includes an air inlet, an air flow controller, a carbon-based material, and an air outlet, wherein the carbon-based material is positioned between the air inlet and the air outlet. In some further embodiments of the invention, the air filtration system includes a filter positioned between the air inlet and the air outlet. In some even further embodiments, the air filtration system further includes a plurality of electrodes, a plurality of wires, a power source, and a switch, wherein the plurality of electrodes, plurality of wires, power source, and switch are operatively connected to the carbon-based material, to form a carbon-based heater. In some further embodiments, the carbon-based heater can reach a pathogen inactivation temperature when a voltage is applied to the carbon-based material. In certain embodiments, the carbon-based material may include a carbon veil. In other embodiments, the carbon-based material may include a perforated carbon nanotube sheet.

### BRIEF DESCRIPTION OF THE DRAWINGS

The accompanying drawings, which are incorporated in and constitute a part of this specification, illustrate embodiments of the invention and, together with the general description of the invention given above, and the detailed description given below, serve to explain the principles of the invention. Similar reference numerals are used to indicate similar features throughout the various figures of the drawings.

FIG. 1 is a perspective view of a piece of personal protective equipment being worn by an individual.

FIG. 2A is a front view of an apparatus including a piece of personal protective equipment and a carbon-based material in accordance with principles of the present invention.

FIG. 2B is a cross-sectional view of the apparatus of FIG. 2A.

FIG. 2C is a cross-sectional view of an alternate embodiment of an apparatus including PPE and a carbon-based material in accordance with the principles of the present invention.

FIG. 3A is a front view of an apparatus including a piece of personal protective equipment and a carbon-based material internal to the PPE.

FIG. 3B is a cross-sectional view of the apparatus of FIG. 3A.

FIG. 3C is a cross-sectional view of an alternate embodiment of an apparatus including PPE and a carbon-based material in accordance with the principles of the present invention.

FIG. 4 is a front-facing view of an embodiment of a carbon-based heater including a carbon veil according to the principles of the present invention.

FIG. 5 is an exploded perspective view of an alternate embodiment of a carbon-based heater including a carbon veil and insulator layer according to the principles of the present invention.

FIG. 6A is a perspective view of an embodiment of a carbon-based heater including at least one CNT sheet according to the principles of the present invention.

FIG. 6B is a perspective view of an alternate embodiment of a carbon-based heater including a plurality of cross-ply CNT sheets according to the principles of the present invention.

FIG. 6C is a perspective view of another embodiment of a carbon-based heater including a plurality of cross-ply CNT sheets according to the principles of the present invention.

FIG. 7 is a front-facing view of an apparatus including a piece of personal protective equipment and a cross-ply CNT sheet according to the principles of the present invention.

FIG. 8A is a perspective view of an embodiment of an insulating layer with holes having a cross-section with a circle shape according to principles of the present invention.

FIG. 8B is a perspective view of an embodiment of an insulating layer with holes having a cross-section with a square shape according to principles of the present invention.

FIG. 9 is a schematic view of an embodiment of the carbon-based heater used in a system for inactivating and removing pathogens from the air of an enclosed space according to the principles of the present invention.

FIG. 10 is a graph showing time dependence of the average temperature on the surface of the commercial ASTM level 3 mask configured to be proximal to the user's face when worn while the carbon-based heater is turned on. The commercial ASTM level 3 mask further includes a nylon insulating layer between the carbon-based heater, including a carbon veil, and the side of the mask configured to be proximal to the user's face when worn.

FIG. 11 is a graph showing time dependence of the average temperature on the surface of the commercial ASTM level 3 mask configured to be proximal to the user's face when worn while the carbon-based heater is turned on. The commercial ASTM level 3 mask further includes an insulating layer between the carbon-based heater, including a carbon veil, and the side of the mask configured to be proximal to the user's face when worn.

FIG. 12 is a graph showing time dependence of the average temperature on the surface of the commercial ASTM level 3 mask configured to be proximal to the user's face when worn while the carbon-based heater is turned on. The commercial ASTM level 3 mask further includes an insulating layer between the carbon-based heater, including a carbon veil, and the side of the mask configured to be proximal to the user's face when worn.

FIG. 13 is a graph showing the pressure drop across a piece of personal protective equipment including (1) a carbon-based heater including carbon veil and an ASTM level 3 mask; (2) a carbon-based heater including carbon veil, a 1.2 mm thick 3D printed nylon insulating layer with a square holes having a side length of 2 mm, and an ASTM level 3 mask; and (3) a carbon-based heater including carbon veil, a 2.1 mm thick 3D printed nylon insulating layer with a square holes having a side length of 2 mm, and an ASTM level 3 mask.

FIG. 14 is a graph showing the pressure drop across a piece of personal protective equipment including (1) a carbon-based heater including carbon veil and an ASTM level 3 mask; (2) a carbon-based heater including carbon veil, a 2 mm thick 3D printed nylon insulating layer with a square holes having a side length of 5 mm, and an ASTM level 3 mask; and (3) a carbon-based heater including carbon veil, a 2.9 mm thick 3D printed nylon insulating layer with a square holes having a side length of 5 mm, and an ASTM level 3 mask.

### DETAILED DESCRIPTION OF EMBODIMENTS OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described above, one aspect of the present invention is directed to an apparatus that provides enhanced filtration of pathogens and/or other methods of trapping, reducing, or eliminating pathogens—and thus provide enhanced protection to an individual. One embodiment of such an apparatus may include a piece of personal protective equipment and a carbon-based material. The piece of personal protective equipment may be a mask, such as one that may be typically worn to prevent pathogens from entering a body via the nose or mouth. Certain such masks include an inner layer and an outer layer, and in various embodiments of the present invention, the carbon-based material may be disposed relative to the mask at a location selected from an external surface of the inner layer, an external surface of the outer layer, and between the inner and outer layers. In some embodiments, the carbon-based material may include a carbon veil. In some embodiments, the carbon-based material includes a carbon nanotube sheet. The presence of the carbon-based material may provide a filtration function to the apparatus (or may provide an additional or enhanced filtration function to a level of filtration that is already provided by the layer or layers of the mask itself. And another aspect of the present invention is directed to an apparatus that includes a heating function to reduce or eliminate pathogens—and thus provide enhanced protection to an individual. Some embodiments in accordance with this aspect of the invention may include the apparatus (i.e., personal protective equipment with carbon-based material) described above. In certain embodiments, the apparatus further includes a plurality of electrodes, a plurality of wires, a power source, and a switch, wherein the plurality of electrodes, plurality of wires, power source, and switch are operatively connected to the carbon-based material, to form a carbon-based heater. The carbon-based heater can reach a pathogen inactivation threshold temperature when a voltage is applied to the carbon-based material.

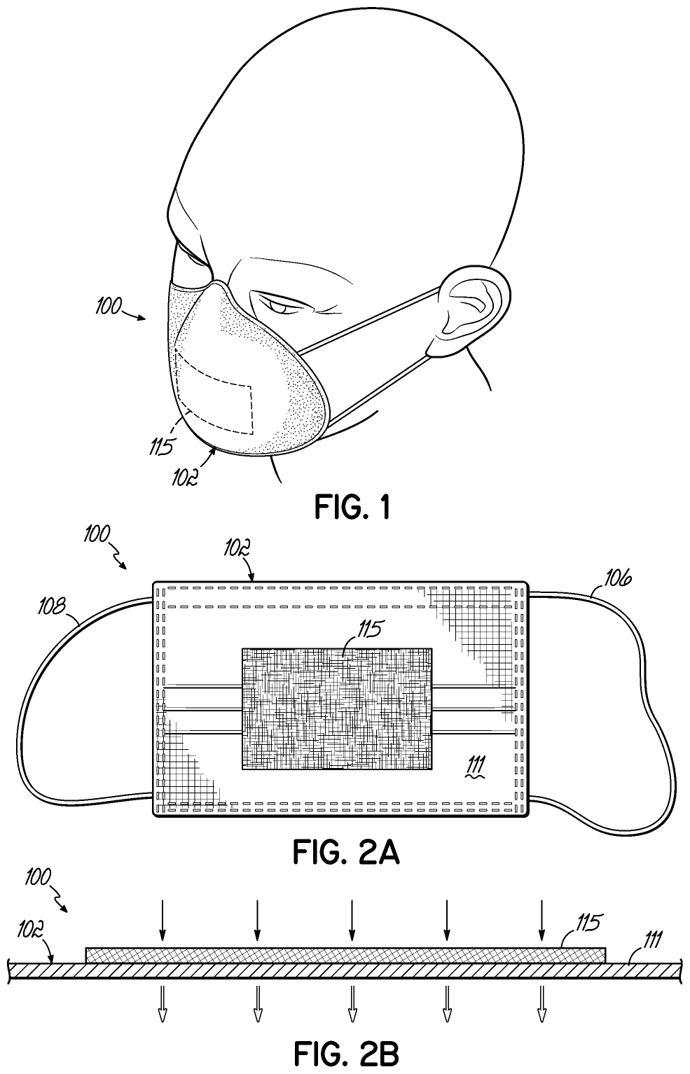

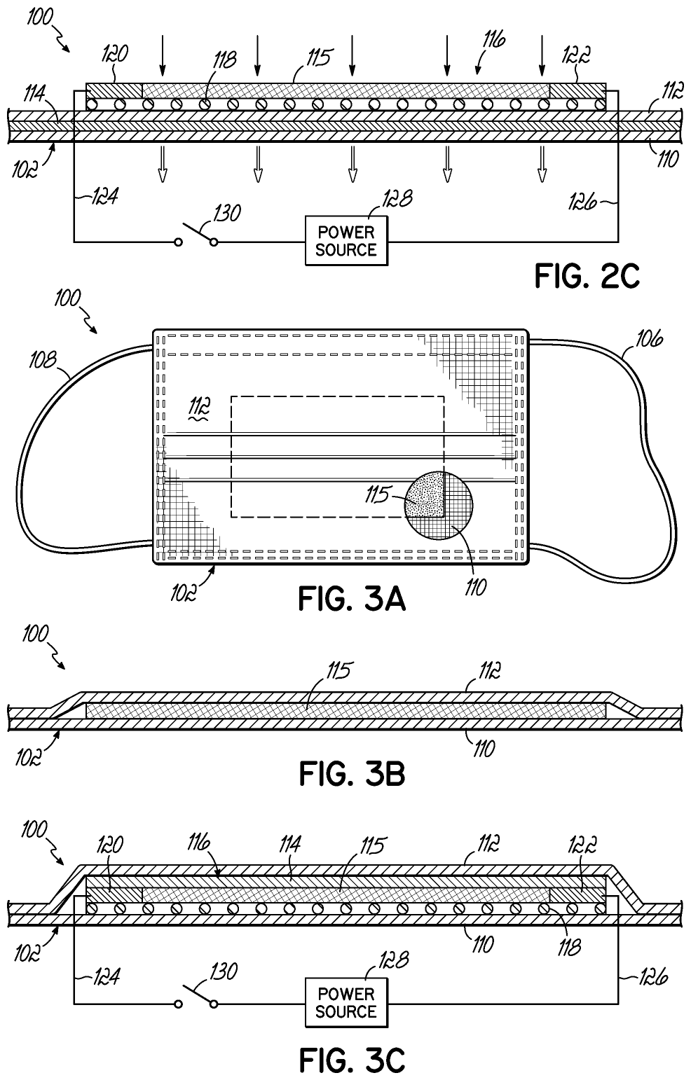

With reference to FIG. 1, an example of an apparatus 100 including a piece of personal protective equipment 102 that may be used in accordance with aspects of the present invention is shown. As shown, the piece of personal protective equipment may be a facemask. In exemplary embodiments, the facemask may include an N95 respirator. In other embodiments, the facemask may be an ASTM level 3 facemask (see FIGS. 2A-3C) or a cloth mask (not shown). With reference to FIG. 1, the piece of personal protective equipment 102 includes straps 106, 108 (strap 108 can be seen in FIG. 2) for securing the piece of personal protective equipment 102 to the face of a user. The straps 106, 108 may be used to secure the piece of personal protective equipment 102 to the face of a user by looping the straps around the ears of a user. Alternatively, the piece of personal protective equipment 102 may be used together with auxiliary equipment that allows the piece of personal protective equipment 102 to be secured to the user's face without looping straps 106, 108 around the ears (not shown).

With reference to FIGS. 2A-3C, it can be seen that those illustrated embodiments of the apparatus 100 include multiple layers. When referring to these depictions, a layer depicted and/or described as above another layer is configured to be more exterior relative to a user's face when worn than the other layer. Similarly, a layer depicted and/or described as below another layer is configured to be more interior relative to a user's face when worn than the other layer. When a layer is described relative to another layer, the term “between” is understood to mean “in the space separating at least those two other layers, but not necessarily in physical contact with either of those layers.” When a layer is described relative to another layer, the term “above” means “configured to be more exterior than at least that other layer, but not necessarily in physical contact with that other layer.” When a layer is described relative to another layer, the term “below” means “configured to be more interior than at least that other layer, but not necessarily in physical contact with that other layer.” When any of the terms “between,” “above,” or “below,” are modified by directly (e.g., directly between), then the layer must be in physical contact with at least a portion of the other layer.

With reference to FIGS. 2A-2B, the apparatus 100 includes a piece of personal protective equipment 102 and a carbon-based material 115. The piece of personal protective equipment 102 includes, for example, an ASTM level 3 facemask with a mask layer 111. As shown, the carbon-based material 115 is directly above the mask layer 111. The piece of personal protective equipment 102 includes straps 106, 108 that may be used to secure the piece of personal protective equipment 102 to the face of a user by looping the straps around the ears of a user (not shown). Alternatively, the piece of personal protective equipment 102 may be used together with auxiliary equipment that allows the piece of personal protective equipment 102 to be secured to the user's face without looping the straps 106, 108 around the ears (not shown).

With reference to FIG. 2C, another embodiment of the apparatus 100 including the piece of personal protective equipment 102 and carbon-based material 115 is shown. As shown, the piece of personal protective equipment 102 includes an inner mask layer 110 configured to be proximal to a user's face (not shown) when worn. Above the inner mask layer 110 is the outer mask layer 112. Between the inner mask layer 110 and the outer mask layer 112 may be an optional filter layer 114. In some embodiments, the optional filter layer 114 is directly above the inner mask layer 110 and directly below the outer mask layer 112. A carbon-based heater 116, which includes the carbon-based material 115, is positioned above the outer mask layer 112. As shown in FIG. 2C, the carbon-based heater 116 further includes a first electrode 120 and a second electrode 122 which are attached to a first wire 124 and a second wire 126 respectively. Wires 124, 126 are further connected to a power source 128 which in turn is connected to a switch 130. Although the switch 130 is shown on first wire 124, embodiments of the invention not shown here include the switch 130 on the second wire 126 (and switch 130 ultimately may be placed anywhere that allows carbon-based heater 116 to be operable). As shown, an optional insulating layer 118 is positioned between the outer mask layer 112 and the carbon-based heater 116. In some embodiments, the optional insulating layer 118 is positioned directly above the outer mask layer 112 and directly below the carbon-based heater 116. In some further embodiments, the inner mask layer 110 is directly below the optional filter layer 114, the optional filter layer 114 is directly below the outer mask layer 112, the outer mask layer 112 is directly below the optional insulating layer 118, and the optional insulating layer 118 is directly below the carbon-based heater 116.

Turning now to FIGS. 3A-3C, an alternate embodiment of the apparatus 100 including the piece of personal protective equipment 102 and the carbon-based material 115 is shown. With reference to FIGS. 3A and 3B, the piece of personal protective equipment 102 includes an ASTM level 3 facemask with the inner mask layer 110 and outer mask layer 112. As shown, the carbon-based material 115 is positioned directly between the inner mask layer 110 and the outer mask layer 112. The piece of personal protective equipment 102 includes straps 106, 108 that may be used to secure the piece of personal protective equipment 102 to the face of a user by looping the straps around the ears of a user (not shown). Alternatively, the piece of personal protective equipment 102 may be used together with auxiliary equipment that allows the piece of personal protective equipment 102 to be secured to the user's face without looping the straps 106, 108 around the ears (not shown).

With reference to FIG. 3C, an alternate embodiment of the apparatus 100 including the piece of personal protective equipment 102 and the carbon-based material 115 is shown. As shown, the piece of personal protective equipment 102 includes the inner mask layer 110 configured to be proximal to a user's face (not shown) when worn. Above the inner mask layer 110 is the outer mask layer 112. Between the inner mask layer 110 and the outer mask layer 112 may be the optional filter layer 114. In some embodiments, the optional filter layer 114 is directly below the outer mask layer 112. The carbon-based heater 116, including the carbon-based material 115, is positioned below the outer mask layer 112 and above the inner mask layer 110. As shown in FIG. 3C, the carbon-based heater 116 further includes a first electrode 120 and a second electrode 122 which are attached to a first wire 124 and a second wire 126 respectively. Wires 124, 126 are further connected to a power source 128 which in turn is connected to a switch 130. Although the switch 130 is shown on first wire 124, embodiments of the invention not shown here include the switch 130 on the second wire 126 (and switch 130 ultimately may be placed anywhere that allows carbon-based heater 116 to be operable). In some embodiments, the carbon-based heater 116 is positioned directly above the inner mask layer 110. In other embodiments, the optional insulating layer 118 may be positioned directly between the carbon-based heater 116 and the inner mask layer 110. In some embodiments, the inner mask layer 110 is directly below the optional insulating layer 118, the optional insulating layer 118 is directly below the carbon-based heater 116, the carbon-based heater 116 is directly below the optional filter layer 114, and the optional filter layer 114 is directly below the outer mask layer 112.

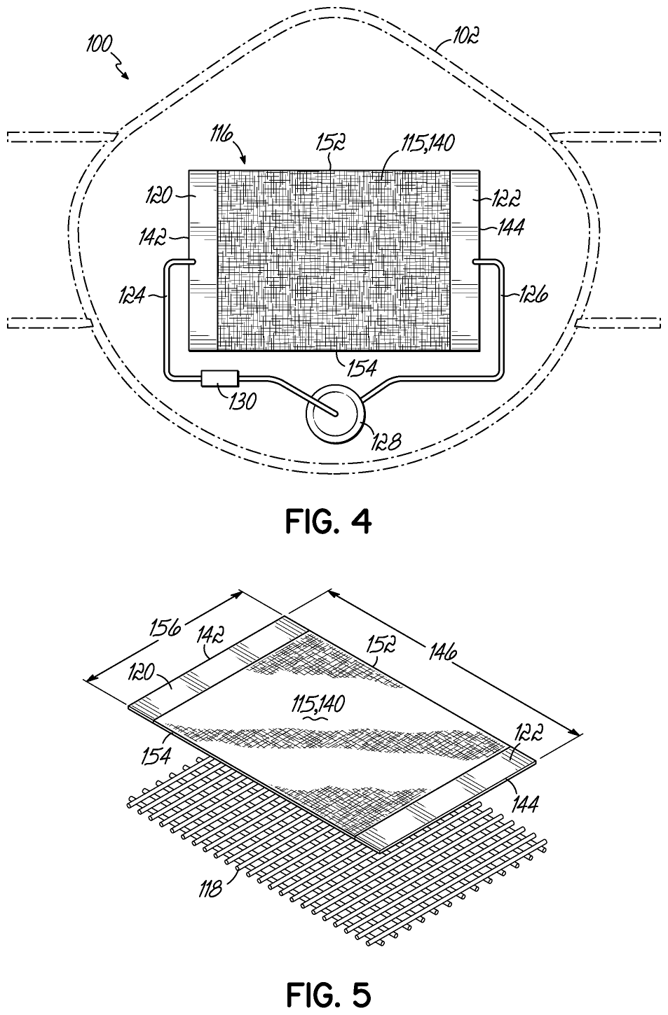

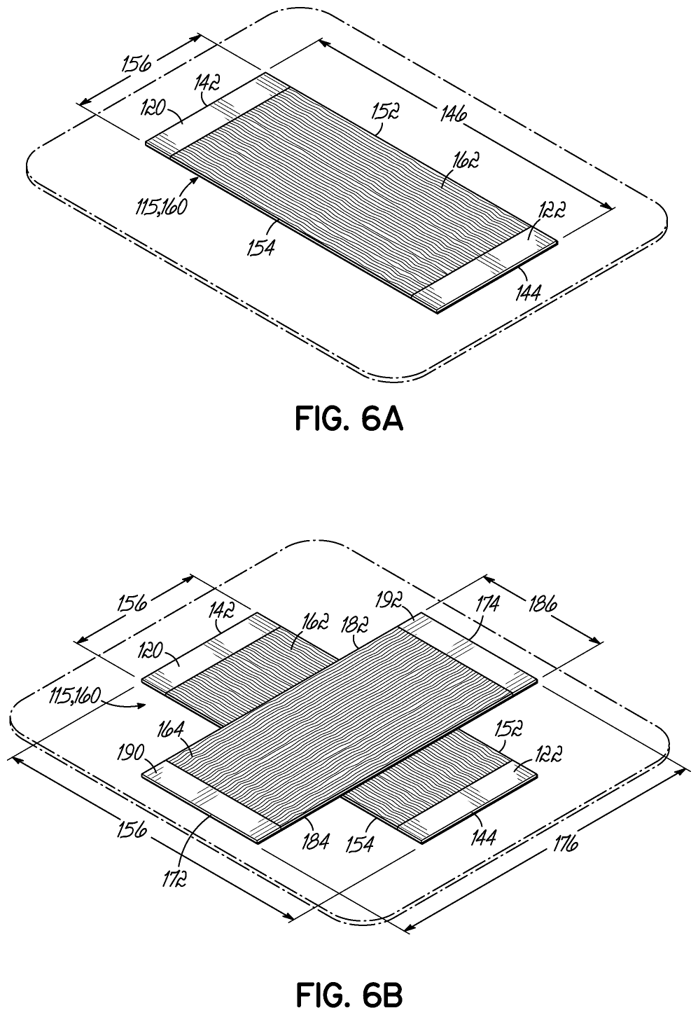

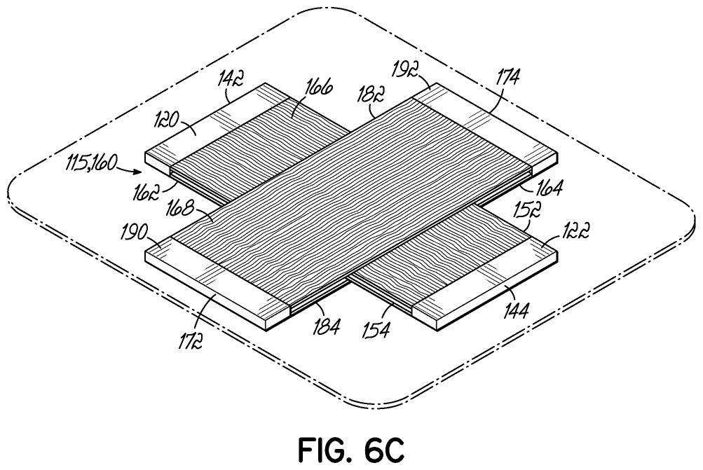

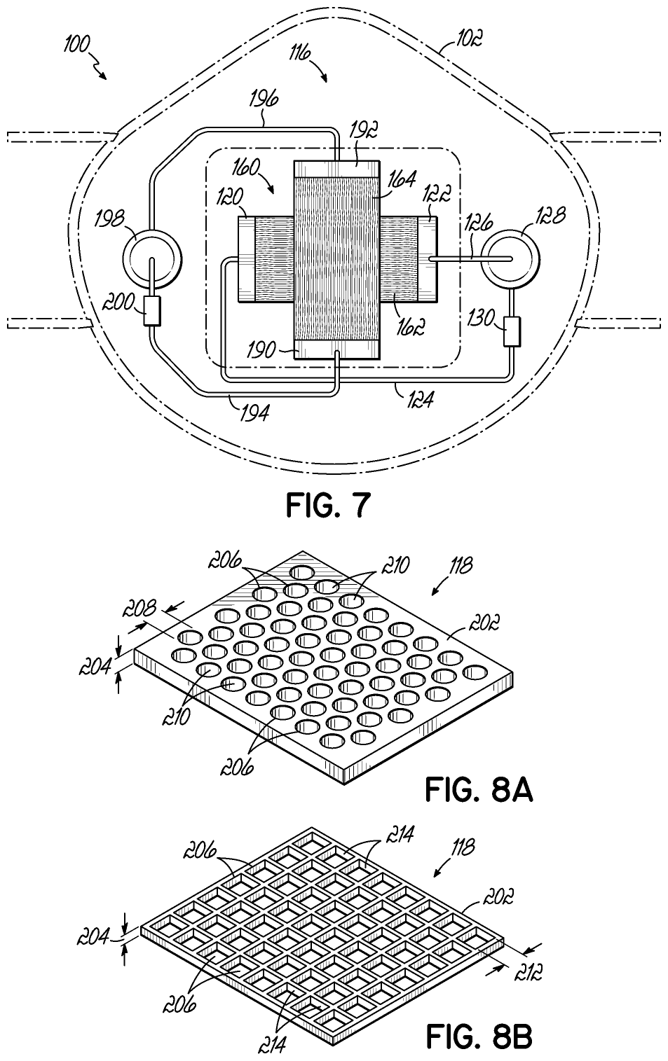

With reference to the embodiments described above for FIGS. 2A-3C, the carbon-based material 115 may include one or more materials such as, by way of example and not limitation, a carbon veil 140 (see FIGS. 4 and 5), at least one carbon nanotube (CNT) sheet 160 (see FIGS. 6A-7), bucky paper, graphene, or some other non-metallic heatable material. The carbon-based material 115 may also serve the function of providing a tortuous path for pathogens to traverse before the user of the apparatus 100 is exposed to the pathogens.

In embodiments utilizing carbon veil 140 (See FIGS. 4 and 5) as the carbon-based material 115, the carbon veil 140 may include chopped carbon fibers which are consolidated in the form of a non-woven fabric. This non-woven fabric may be held together by binder compounds, including but not limited to polymers such as acrylonitrile styrene acrylate. The heated area generated by the carbon veil 140 varies depending on the piece of personal protective equipment 102. In some embodiments, the heated area is greater than or equal to 12 cm² and less than or equal to 150 cm². In other embodiments, the heated area is greater than or equal to 25 cm² and less than or equal to 100 cm². The average dimensions of the heated area may be approximately 100 cm². The carbon veil 140 has a thickness that can vary between different embodiments. In some embodiments, the carbon veil 140 has a thickness greater than or equal to 0.1 micron and less than or equal to 3 mm. In other embodiments, the carbon veil has a thickness greater than or equal to 0.05 mm and less than or equal to 0.127 mm.

In embodiments utilizing a carbon-based material 115 with at least one CNT sheet 160 (See FIGS. 6A-7), the CNT sheets 160 include spinnable CNT arrays that are drawn into aligned sheets. Carbon-based heaters 116 using CNT sheets 160 exhibit ultrafast thermal response over 1000° C./s, low operation voltage, long lifetime, and lighter weight than metallic heaters. In some embodiments, a plurality of CNT sheets 160 may be arranged in cross-ply multi-layers by orienting the adjacent CNT sheets perpendicularly relative to each other. The total number of CNT sheets 160 used in the carbon-based material 115 may vary. In some embodiments, the carbon-based material 115 may include greater than or equal to 10 CNT sheets 160 and less than or equal to 100 CNT sheets 160. In other embodiments, the carbon-based material 115 may include greater than or equal to 1 CNT sheet 160 and less than or equal to 1,000 CNT sheets 160. The total heated area of the carbon-based material 115 including at least one CNT sheet 160 varies depending on the piece of personal protective equipment 102. In some embodiments, the total heated area is greater than or equal to 12 cm² and less than or equal to 150 cm². In other embodiments, the heated area is greater than or equal to 25 cm² and less than or equal to 100 cm². In some embodiments, the average dimensions of the heated area may be approximately 25 cm².

Applying a voltage to the carbon-based heater 116 generates Ohmic heating, adding an additional protective layer to the apparatus 100 in the form of a heat shield that provides a thermal barrier for airborne pathogens and enables their inactivation. In order to inactivate a pathogen, the temperature of the carbon-based heater 116 may be raised to a pathogen inactivation threshold temperature. This pathogen inactivation threshold temperature may depend on the pathogen to be inactivated. For some pathogens, including SARS-CoV-2, the pathogen inactivation temperature is at least 65° C. Other pathogens that may be inactivated using heat include, by way of example and not limitation, coronaviruses, tuberculosis bacteria (Mycobacterium tuberculosis), human immunodeficiency virus (HIV), Ebola virus, biological weapons, and other organic airborne contaminants. The voltage needed to raise the temperature of the carbon-based heater 116 to the pathogen inactivation threshold depends on the size of the carbon-based heater 116 used. Generally speaking, the larger the area of the carbon-based heater 116, the larger voltage is required to reach the pathogen inactivation threshold temperature. In some embodiments, the pathogen inactivation threshold temperature can be reached by applying a voltage greater than or equal to 0.3 V and less than or equal to 50 V. In some further embodiments, the pathogen inactivation threshold temperature can be reached by applying a voltage greater than or equal to 3 V and less than or equal to 17 V. In some even further embodiments, the pathogen inactivation threshold temperature can be reached by applying a voltage greater than or equal to 3 V and less than or equal to 9 V.

The optional insulating layer 118 may be included to attenuate the heat from the heater to the face skin of a user. Any conventional material for heat insulation may be used in the optional insulating layer 118 including, by way of example and not limitation, low thermal conductivity polymers, fiberglass, polylactic acid (PLA), polyhydroxyalkanoate (PHA), cellulose, polyester, starch, polyvinyl alcohol (PVA), natural silk, natural wood, silica aerogels, alumina aerogels, or other suitable insulators. The optional insulating layer 118 may be configured to include air pockets between the heater and the face skin. The optional insulating layer 118 may be 3D printed using polymer filaments. In such embodiments, the optional insulating layer 118 may be 3D printed to include holes with a cross section of a standardized shape and a standardized size. The optional insulating layer 118 also has a thickness that may be greater than or equal to 0.1 and less than or equal to 5 mm. In some further embodiments, the insulating layer 118 has a thickness greater than or equal to 0.1 mm and less than or equal to 3 mm. In some even further embodiments, the optional insulating layer 118 has a depth that is greater than or equal to 0.8 mm and less than or equal to 2.9 mm. In some even further embodiments, the optional insulating layer 118 has a depth that is greater than or equal to 1 mm and less than or equal to 2 mm. In another embodiment, the optional insulating layer 118 has a thickness greater than or equal to 1 mm and less than or equal to 6 mm. In some embodiments, the optional insulating layer 118 includes a separate thermally insulating membrane and a spacer.

The electrodes 120, 122 may include a metal configured to convey current from the power source 128 to the carbon-based heater 116. The electrodes 120, 122 may include metals such as, by way of example and not limitation, copper, silver, gold, aluminum, and other metals. The electrodes 120, 122 may be added to the carbon-based heater 116 in several ways including, by way of example and not limitation, electrodeposition, metal tape, physical vapor deposition (e.g., electron beam evaporation, thermal evaporation, and magnetron sputtering), and other similar methods for affixing a metal to the carbon-based material 115. Electrodeposition of the electrode 120, 122 may be enabled or facilitated via pretreatment of the carbon-based material 115 with atmospheric pressure helium or oxygen plasma, which functionalizes the exposed areas, facilitates wetting the carbon-based material 115 in the metallic salt solution for electrodeposition, and allows the electrode 120, 122 to adhere uniformly to the carbon-based material 115.

The wires 124, 126 may include a metal configured to convey current from the power source 128 to the carbon-based heater 116. The wires 124, 126 may include metals such as, by way of example and not limitation, copper, silver, gold, aluminum, and other metals. The wires 124, 126 may be attached to the electrodes 120, 122 respectively by a conventional method of attachment including, by way of example and not limitation, soldering, conductive metal paste like silver or nickel paste, carbon paste, bonding via compression combined with heat in a reducing gas environment like hydrogen, clamping, crimping, any electrically conductive glue, or any other conventional method of bonding metals together.

The power source 128 may be implemented as an internal power source 128 provided by any conventional portable source of electricity including, by way of example and not limitation, a battery. In some embodiments, a plurality of batteries in series is used to provide a voltage sufficient to reach the pathogen inactivation threshold temperature. The internal power source 128 may be removably attached to the piece of personal protective equipment 102 using Velcro. Alternatively, the piece of personal protective equipment 102 may include a pocket configured to receive the internal power source 128. In other embodiments, the power source 128 may be implemented as a connector configured to receive power from an external power source (not shown) including, by way of example and not limitation, a phone or wall outlet.

The switch 130 may be used to control the thermal response of the apparatus 100. In some embodiments, the heat response of the switch 130 and the carbon-based heater 116 are quick enough to reach the pathogen inactivation threshold temperature from an off position within a few seconds. In some embodiments, the heat response of the switch 130 and the carbon-based heater 116 are quick enough to reach room temperature from an on position within a few seconds. The switch 130 may be implemented by conventional methods of completing a circuit including, by way of example and not limitation, a microswitch.

With reference to FIGS. 4 and 5, an embodiment of the carbon-based heater 116 including a carbon-based material 115, wherein the carbon-based material is the carbon veil 140, is shown. As shown in FIG. 4, the carbon veil 140 has a rectangular shape with a first end 142 and a second end 144 opposite the first end 142, where the first end 142 and the second end 144 are separated by a first distance 146. The carbon veil 140 further includes a third end 152 and a fourth end 154 opposite the third end 152, where the third end 152 and the fourth end 154 are separated by a second distance 156. In some embodiments, the first distance 146 is greater than the second distance 156. In some embodiments, the carbon veil 140 is configured to be oriented relative to the piece of personal protective equipment 102 such that the first end 142 and the second end 144 are oriented right-left or left-right relative to the user's face when worn (not shown). In other embodiments, the carbon veil 140 is oriented relative to the piece of personal protective equipment 102 such that the first end 142 and the second end 144 are oriented up-down or down-up relative to the user's face when worn (not shown). As shown, the first end 142 is covered by first electrode 120 and the second end 144 is covered by the second electrode 122. Electrodes 120, 122 are connected by wires 124, 126 respectively to a power source 128 which in turn is connected to a switch 130. The power source 128 may be implemented as at least one battery attached to the outer mask layer 112 using a conventional method of attachment such as Velcro or by adding a pocket to the outer mask layer 112. In other embodiments, the power source 128 is a connector configured to receive power from an external source such as a cell phone or an outlet (not shown). Although the switch 130 is shown on first wire 124, embodiments of the invention not shown here include the switch 130 on the second wire 126. While the carbon veil 140 is shown to have a rectangular shape, other embodiments not shown here have different shapes including, by way of example and not limitation, a circle, a semi-circle, trapezoid, rhombus, triangle, square, hexagon, or other suitable shapes. With reference to FIG. 4, an exploded view of the carbon-based heater 116 including a carbon veil 140 is shown. As shown, the carbon veil 140 may be separated from the outer mask layer 112 by the optional insulating layer 118.

With reference to FIGS. 4 and 5, the carbon-based heater 116 may include a carbon veil 140 with multiple layers (not shown). In such embodiments, the electrodes 120, 122 may be connected to all layers of the carbon veil 140. Further, the multiple layers of the carbon veil 140 may increase the thickness of the carbon-based heater 116, which in turn may increase the pressure drop across the apparatus 100. In some embodiments, the carbon-based heater 116 may include a carbon veil 140 which is perforated (not shown). In such embodiments, the perforation of the carbon veil 140 may decrease the pressure drop increase caused by the carbon veil 140.

With reference to FIGS. 6A-6C, embodiments of the carbon-based heater 116 including the carbon-based material 115, wherein the carbon-based material 115 includes at least one CNT sheet 160 with a first CNT sheet 162, is shown. As shown in FIG. 6A, the first CNT sheet 162 has a rectangular shape with a first end 142 and a second end 144 opposite the first end 142, where the first end 142 and the second end 144 are separated by a first distance 146. The first CNT sheet 162 further includes a third end 152 and a fourth end 154 opposite the third end 152, where the third end 152 and the fourth end 154 are separated by a second distance 156. In some embodiments, the first distance 146 is greater than the second distance 156. In some embodiments, the first CNT sheet 162 is configured to be oriented relative to the piece of personal protective equipment 102 such that the first end 142 and the second end 144 are oriented right-left or left-right relative to the user's face when worn (see FIG. 7). In other embodiments, the first CNT sheet 162 is oriented relative to the piece of personal protective equipment 102 such that the first end 142 and the second end 144 are oriented up-down or down-up relative to the user's face when worn (not shown). As shown, the first end 142 is covered by first electrode 120 and the second end 144 is covered by the second electrode 122. While the first CNT sheet 162 is shown to have a rectangular shape, other embodiments not shown here have different shapes including, by way of example and not limitation, a circle, a semi-circle, trapezoid, rhombus, triangle, square, hexagon, or other suitable shapes.

With further reference to FIG. 6A, the carbon-based heater 116 may include a plurality CNT sheets 160 (not shown). In such embodiments, the electrodes 120, 122 may be connected to all layers of the CNT sheet 160. Further, the plurality of CNT sheets 160 may increase the thickness of the carbon-based heater 116, which in turn may increase the pressure drop across the apparatus 100. In some embodiments, the carbon-based heater 116 may include at least one CNT sheet 160 which is perforated (not shown). In such embodiments, the perforation of the at least one CNT sheet 160 may decrease the pressure drop increase caused by the at least one CNT sheet 160.

With reference to FIG. 6B, a further embodiment of the carbon-based heater 116 including a plurality of cross-ply CNT sheets 160 is shown. As shown, the plurality of cross-ply CNT sheets 160 includes at least the first CNT sheet 162 and a second CNT sheet 164 where the second CNT sheet 164 is oriented perpendicularly relative to the first CNT sheet 162. In some further embodiments, the CNT sheets 162, 164 are oriented relative to the piece of personal protective equipment 102 such that the plurality of cross-ply CNT sheets 160 resembles an addition sign. In other further embodiments, the CNT sheets 162, 164 are oriented relative to the piece of personal protective equipment 102 such that the plurality of cross-ply CNT sheets 160 resembles a letter “x”. The second CNT sheet 164 has a first end 172 and a second end 174 opposite from the first end 172, where the first end 172 and second end 174 are separated by a first distance 176. The second CNT sheet 164 has a third end 182 and a fourth end 184 opposite from the third end 182, where the third end 182 and fourth end 184 are separated by a second distance 186. In some embodiments, the first distance 176 is greater than the second distance 186. As shown, the second CNT sheet 164 includes a third electrode 190 configured to cover the first end 172 and a fourth electrode 192 configured to cover the second end 174.

With reference to FIG. 6C, another further embodiment of the carbon-based heater 116 including a plurality of cross-ply CNT sheets 160 is shown. As shown, the plurality of cross-ply CNT sheets 160 further includes a third CNT sheet 166 and a fourth CNT sheet 168. The third CNT sheet 166 has the same components as described in first CNT sheet 162 above. The fourth CNT sheet 168 has the same components as described in the second CNT sheet 164 above. As shown, the third CNT sheet 166 is oriented to be in line with the first CNT sheet 162 and the fourth CNT sheet 168 is oriented to be in line with the second CNT sheet 164. In some embodiments, the first CNT sheet 162 and third CNT sheet 166 have multiple first ends 142 that are connected by a single first electrode 120 and multiple second ends 144 that are connected by a single second electrode 122. Further, the second CNT sheet 164 and fourth CNT sheet 168 have multiple first ends 172 that are connected by a single third electrode 190 and multiple second ends 174 that are connected by a single fourth electrode 192. Although four CNT sheets 162, 164, 166, 168 are shown, a greater plurality of layers may be used (not shown). In such embodiments, odd numbered CNT sheets may be oriented to be in line with first CNT sheet 162 and third CNT sheet 166 and include the same components described in the first CNT sheet 162 above. Further, in such embodiments, even numbered CNT sheet may be oriented to be in line with second CNT sheet 164 and fourth CNT sheet 168 and include the same components described in the second CNT sheet 164 above. Further, the plurality of CNT sheets 160 may increase the thickness of the carbon-based heater 116, which in turn may increase the pressure drop across the piece of personal protective equipment 102. In some embodiments, the carbon-based heater 116 may include a plurality of CNT sheets 160 which are perforated (not shown). In such embodiments, the perforation of the plurality CNT sheets 160 may decrease the pressure drop increase caused by the plurality of CNT sheets 160. While each of the plurality of CNT sheets 160 are shown to have a rectangular shape, other embodiments not shown here have different shapes including, by way of example and not limitation, a circle, a semi-circle, trapezoid, rhombus, triangle, square, hexagon, or other suitable shapes.

With reference to FIG. 7, a further embodiment of the apparatus 100 including the piece of personal protective equipment 100 and the carbon-based heater 116 including the plurality of cross-ply CNT sheets 160 demonstrated in FIG. 6C is shown relative to the piece of personal protective equipment 102. As shown, the carbon-based heater 116 further includes the first wire 124 connected to the first electrode 120, the second wire 126 connected to the second electrode 122, a third wire 194 connected to the third electrode 190, and a fourth wire 196 connected to the fourth electrode 192. The first wire 124 and second wire 126 are connected to a first power source 128 and the third wire 194 and the fourth wire 196 are connected to a second power source 198. The first power source 128 is controlled by a first switch 130 and the second power source 198 is controlled by a second switch 200. Although not shown, embodiments of invention may include only a single power source. Although not shown, embodiments of the invention may have at least one power source that is controlled by a single switch. The power sources 128, 198 may each be implemented as at least one battery attached to the outer mask layer 112 using a conventional method of attachment such as Velcro or by adding a pocket to the outer mask layer 112 (not shown). In other embodiments, the power sources 128, 198 are each implemented as a connector configured to receive power from an external source such as a cell phone or an outlet (not shown). Although the switch 130 is shown on first wire 124, embodiments of the invention not shown here include the switch 130 on the second wire 126. Similarly, although the switch 200 is shown on third wire 194, embodiments of the invention not shown here include the switch 200 on the fourth wire 196. Although not visible in FIG. 7, the optional insulating layer 118 may be situated between the piece of personal protective equipment 102 and the carbon heater 116.

With reference to FIG. 7, the carbon-based heater 116 may include a plurality of cross-ply CNT sheets 160. In such embodiments, the electrodes 120, 122, 190, 192 may be connected to each of the CNT sheets 162, 164, 166, 168 of the plurality of cross-ply CNT sheets 160 oriented in the same direction. Although four CNT sheets 162, 164, 166, 168 are shown, a greater plurality of CNT sheets 160 may be used (not shown). In such embodiments, odd numbered CNT sheets may be oriented to be in line with first CNT sheet 162 and third CNT sheet 166 and include the same components described in the first CNT sheet 162 above. Further, in such embodiments, even numbered CNT sheets may be oriented to be in line with second CNT sheet 164 and fourth CNT sheet 168 and include the same components described in the second CNT sheet 164 above. Further, the plurality of cross-ply CNT sheets 160 may increase the thickness of the carbon-based heater 116, which in turn may increase the pressure drop across the apparatus 100. In some embodiments, the carbon-based heater 116 may include a plurality of cross-ply CNT sheets 160 which are perforated (not shown). In such embodiments, the perforation of the plurality of cross-ply CNT sheets 160 may decrease the pressure drop increase caused by the plurality of cross-ply CNT sheets 160.

With reference to FIG. 8A, an embodiment of the optional insulating layer 118 including a 3D printed spacer 202 is shown. As shown, the 3D printed spacer 202 has a thickness 204 and a plurality of holes 206. Each of the plurality of holes 206 has a cross-section with a circular shape having a diameter 208. Accordingly, each of the plurality of holes 206 include at least one curved sidewall 210. Although not shown, each of the plurality of holes 206 may have a cross-section with a different shape including, by way of example and not limitation, a semicircle, or any other shape capable of being repeated across a 2D plane.

With reference to FIG. 8B, an embodiment of the optional insulating layer 118 including a 3D printed spacer 202 is shown. As shown, the 3D printed spacer 202 has a thickness 204 and a plurality of holes 206. Each of the plurality of holes 206 has a cross-section with a square shape having a side length 212. Accordingly, each of the plurality of holes 206 includes at least one flat sidewall 214. Although not shown, each of the plurality of holes 206 may have a cross-section with a different shape including, by way of example and not limitation, a triangle, a hexagon, or any other suitable polygon capable of being repeated across a 2D plane.

With reference to the embodiments described above for FIGS. 4-8B, applying a voltage to the carbon-based heater 116 generates Ohmic heating, adding an additional protective layer to the apparatus 100 in the form of a heat shield that provides a thermal barrier for airborne pathogens and enables their inactivation. In order to inactivate a pathogen, the temperature of the carbon-based heater 116 may be raised to a pathogen inactivation threshold temperature. This pathogen inactivation threshold temperature may depend on the pathogen to be inactivated. For some pathogens, including SARS-CoV-2, the pathogen inactivation temperature is at least 65° C. Other pathogens that may be inactivated using heat include, by way of example and not limitation, coronaviruses, tuberculosis bacteria (Mycobacterium tuberculosis), human immunodeficiency virus (HIV), Ebola virus, biological weapons, and other organic airborne contaminants. The voltage needed to raise the temperature of the carbon-based heater 116 to the pathogen inactivation threshold depends on the size of the carbon-based heater 116 used. Generally speaking, the larger the area of the carbon-based heater 116, the larger voltage is required to reach the pathogen inactivation threshold temperature. In some embodiments, the pathogen inactivation threshold temperature can be reached by applying a voltage greater than or equal to 0.3 V and less than or equal to 50 V. In some further embodiments, the pathogen inactivation threshold temperature can be reached by applying a voltage greater than or equal to 3 V and less than or equal to 17 V. In some even further embodiments, the pathogen inactivation threshold temperature can be reached by applying a voltage greater than or equal to 3 V and less than or equal to 9 V.

In embodiments utilizing carbon veil 140 as the carbon-based material 115, the carbon veil 140 includes chopped carbon fibers which are consolidated in the form of a non-woven fabric. This non-woven fabric may be held together by binder compounds, including but not limited to polymers such as acrylonitrile styrene acrylate. The heated area generated by the carbon veil 140 varies depending on the piece of personal protective equipment 102. In some embodiments, the heated area is greater than or equal to 12 cm² and less than or equal to 150 cm². In other embodiments, the heated area is greater than or equal to 25 cm² and less than or equal to 100 cm². The average dimensions of the heated area may be approximately 100 cm². The carbon veil 140 has a thickness that can vary between different embodiments. In some further embodiments, the carbon veil 140 has a thickness greater than or equal to 0.1 micron and less than or equal to 3 mm. In some embodiments, the carbon veil 140 has a thickness greater than or equal to 0.05 mm and less than or equal to 0.127 mm.

In embodiments utilizing a carbon-based material with at least one CNT sheet 160, the CNT sheets 160 include spinnable CNT arrays that are drawn into aligned sheets. Carbon-based heaters 116 using CNT sheets 160 exhibit ultrafast thermal response over 1000° C./s, low operation voltage, long lifetime, and lighter weight than metallic heaters. In some embodiments, a plurality of CNT sheets 160 may be arranged in cross-ply multi-layers by orienting the adjacent CNT sheets 160 perpendicularly relative to each other. The total number of CNT sheets 160 used in the carbon-based material 115 may vary. In some embodiments, the carbon-based material 115 may include greater than or equal to 10 CNT sheets 160 and less than or equal to 100 CNT sheets 160. In other embodiments, the carbon-based material 115 may include greater than or equal to 1 CNT sheet 160 and less than or equal to 1,000 CNT sheets 160. The total heated area of the carbon-based heater 116 including at least one CNT sheet 160 varies depending on the piece of personal protective equipment 102. In some embodiments, the total heated area is greater than or equal to 12 cm² and less than or equal to 150 cm². In other embodiments, the heated area is greater than or equal to 25 cm² and less than or equal to 100 cm². In some embodiments, the average dimensions of the heated area may be approximately 25 cm².

The optional insulating layer 118 may be included to attenuate the heat from the carbon-based heater 116 to the face skin of the user. Any conventional material for heat insulation may be used in the optional insulating layer 118 including, by way of example and not limitation, low thermal conductivity polymers, fiberglass, polylactic acid (PLA), polyhydroxyalkanoate (PHA), cellulose, polyester, starch, polyvinyl alcohol (PVA), natural silk, natural wood, silica aerogels, alumina aerogels, or other suitable insulators. The optional insulating layer 118 may be configured to include air pockets between the carbon-based heater 116 and the face skin. The optional insulating layer 118 may be 3D printed using polymer filaments. In such embodiments, the optional insulating layer 118 may be 3D printed to include holes with a cross section of a standardized shape and a standardized size. The optional insulating layer 118 also has a thickness that may be greater than or equal to 0.1 and less than or equal to 5 mm. In some further embodiments, the insulating layer 118 has a thickness greater than or equal to 0.1 mm and less than or equal to 3 mm. In some even further embodiments, the optional insulating layer 118 has a thickness that is greater than or equal to 0.8 mm and less than or equal to 2.9 mm. In some even further embodiments, the optional insulating layer 118 has a thickness that is greater than or equal to 1 mm and less than or equal to 2 mm. In another embodiment, the optional insulating layer 118 has a thickness greater than or equal to 1 mm and less than or equal to 6 mm. In some embodiments, the optional insulating layer 118 includes a separate thermally insulating membrane and a spacer.

The electrodes 120, 122 may include a metal configured to convey current from the power source 128 to the carbon-based heater 116. The electrodes 120, 122 may include metals such as, by way of example and not limitation, copper, silver, gold, aluminum, and other metals. The electrodes 120, 122 may be added to the carbon-based heater 116 in several ways including, by way of example and not limitation, electrodeposition, metal tape, physical vapor deposition (e.g., electron beam evaporation, thermal evaporation, and magnetron sputtering), and other similar methods for affixing a metal to the carbon-based material 115. Electrodeposition of the electrode 120, 122 may be enabled or facilitated via pretreatment of the carbon-based material 115 with atmospheric pressure helium or oxygen plasma, which functionalizes the exposed areas, facilitates wetting the carbon-based material 115 in the metallic salt solution for electrodeposition, and allows the electrode 120, 122 to adhere uniformly to the carbon-based material 115.

The wires 124, 126 may include a metal configured to convey current from the power source 128 to the carbon-based heater 116. The wires 124, 126 may include metals such as, by way of example and not limitation, copper, silver, gold, aluminum, and other metals. The wires 124, 126 may be attached to the electrodes 120, 122 respectively by a conventional method of attachment including, by way of example and not limitation, soldering, conductive metal paste like silver or nickel paste, carbon paste, bonding via compression combined with heat in a reducing gas environment like hydrogen, clamping, crimping, any electrically conductive glue, or any other conventional method of bonding metals together.

The power source 128 may be implemented as an internal power source 128 provided by any conventional portable source of electricity including, by way of example and not limitation, a battery. In some embodiments, a plurality of batteries in series is used to provide a voltage sufficient to reach the pathogen inactivation threshold temperature. The internal power source 128 may be removably attached to the piece of personal protective equipment 102 using Velcro. Alternatively, the piece of personal protective equipment 102 may include a pocket configured to receive the internal power source 128. In other embodiments, the power source 128 may be implemented as a connector configured to receive power from an external power source (not shown) including, by way of example and not limitation, a phone or wall outlet.

The switch 130 may be used to control the thermal response of the piece of the apparatus 100. In some embodiments, the heat response of the switch 130 and the carbon-based heater 116 are quick enough to reach the pathogen inactivation threshold temperature from an off position within a few seconds. In some embodiments, the heat response of the switch 130 and the carbon-based heater 116 are quick enough to reach room temperature from an on position within a few seconds. The switch 130 may be implemented by conventional methods of completing a circuit including, by way of example and not limitation, a microswitch.

With reference to FIGS. 2A-8B, the carbon-based material 115 may be perforated. When a carbon-based material 115 is used in the apparatus 100, the gas permeability of the apparatus 100 decreases as the thickness of the carbon-based material 115 increases. According to the requirements of the ASTM International Standard F2100-19, the pressure drop for the apparatus 100 should be below 58.8 Pa/cm². If the carbon-based material 115 would otherwise cause too large of a pressure drop, the gas permeability of the carbon-based material 115 may be increased by perforating the carbon-based material 115 to create at least one hole which increases the breathability (i.e., gas permeability) of the apparatus 100 and reduces the flow resistance increase caused by the carbon-based material 115. The carbon-based material 115 can be perforated using mechanical tools capable of puncturing, cutting, boring, milling, or otherwise mechanically creating a hole in the carbon-based material 115. One such embodiment of a mechanical tool used to perforate the carbon-based material 115 includes a roller with multiple spikes or microneedles having controlled dimensions and separation from other spikes or microneedles. In other embodiments, the carbon-based material 115 may be perforated using non-mechanical methods of removing material including, but not limited to, laser etching, oxygen plasma etching, or chemical etching through a mask. In embodiments where the carbon-based material 115 is perforated, the heating performance of the carbon-based heater 116 may not be adversely affected.

The optional filter 114 may serve as a barrier between the user and pathogens or other particulate matter in the air. This optional filter 114 layer may include, by way of example and not limitation, a conventional fabric filter, a polypropylene (PP) membrane, or any other filter conventionally used in a commercial piece of personal protective equipment.

The carbon-based heater 116 may be attached to the piece of personal protective equipment 102 in several ways. In some embodiments of the invention, the carbon-based heater 116 is attached to the piece of personal protective equipment 102 by conventional methods of joining a material to a fabric, including but not limited to ultrasonic sewing, Velcro™, or hot pressing in the presence of an adhesive. In embodiments where the carbon-based heater 116 is integrated into the surface of a piece of personal protective equipment 102 using hot pressing, the adhesive may include, by way of example and not limitation, polyvinyl alcohol.

The carbon-based heater 116 may be designed to be heated only while the apparatus 100 is not worn. This addresses any concerns about a hot object next to the user's face and eliminates the need for either batteries or the optional insulating layer 118. The principle of operation of such an embodiment is that the user wears the not-powered and not-heated apparatus 100 in a hazardous environment for a reasonable time followed by powering the carbon-based heater 116 after taking the apparatus 100 off the face of the user. The heating of the carbon-based heater 116 after wearing the apparatus 100 secures eradication of all of the pathogens trapped on the apparatus 100 thanks to the activated heating. After a short thermal treatment, the apparatus 100 is ready to be reused. In some embodiments, the apparatus 100 can be reused after heating the carbon-based heater 116 for less than or equal to 60 seconds. In some further embodiments, the apparatus 100 is ready to be reused after heating the carbon-based heater 116 for less than or equal to 30 seconds.

As described above, another aspect of the present invention is directed to an air filtration system that—like the apparatus described above—may include a carbon-based material that can provide a filtration function and/or a heating function. Certain embodiments of the air filtration system include an air flow path that includes an air inlet, an air flow controller, a carbon-based material, and an air outlet, wherein the carbon-based material is positioned between the air inlet and the air outlet. In some further embodiments of the invention, the air filtration system includes a filter positioned between the air inlet and the air outlet. In some even further embodiments, the air filtration system further includes a plurality of electrodes, a plurality of wires, a power source, and a switch, wherein the plurality of electrodes, plurality of wires, power source, and switch are operatively connected to the carbon-based material, to form a carbon-based heater. In some further embodiments, the carbon-based heater can reach a pathogen inactivation temperature when a voltage is applied to the carbon-based material. In certain embodiments, the carbon-based material may include a carbon veil. In other embodiments, the carbon-based material may include at least one perforated carbon nanotube (CNT) sheet.

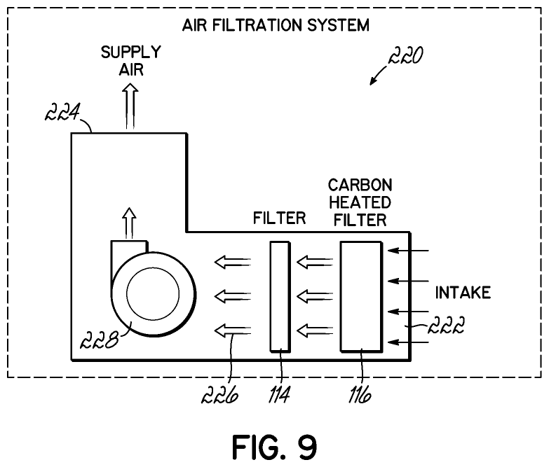

With reference to FIG. 9, an embodiment of an air filtration system 220 used together with the carbon-based material 115 is shown. The air filtration system 220 includes an air inlet 222 and an air outlet 224. Air may be made to flow from the air inlet 222 to the air outlet 224 through air flow path 226 using an air flow controller 228. The air flow controller 228 may be implemented using a device including, by way of example and not limitation, a fan, or vacuum pump, compressor, natural or forced convection, or any other conventional method of causing air to flow preferentially in a given direction. The air flow controller 228 causes air to flow from air inlet 222 to air outlet 224 through air flow path 226. The carbon-based heater 116, which includes the carbon-based material 115 as shown, is configured to cover a cross-section of the air flow path 226 between air inlet 222 and air outlet 224. The carbon-based heater 116 functions both as a filter to trap pathogens and as a heat source to inactivate pathogens. The carbon-based heater 116 includes a first electrode 120 and a second electrode 122, which are connected to the first wire 124 and second wire 126 respectively. The wires 124, 126 are further connected to a power source 128, which in turn is connected to a switch 130. In some embodiments, the air filtration system further includes an optional filter layer 114 that is configured to trap pathogens by covering a cross-section of the air flow path 226 between air inlet 222 and air outlet 224. The air filter 114 may be in contact with the carbon-based heater 116.

The carbon-based material 115 may include one or more materials such as, by way of example and not limitation, the carbon veil 140 (See FIGS. 4 and 5), the at least one carbon nanotube (CNT) sheet (See FIGS. 6A-7), bucky paper, graphene, or some other non-metallic heatable material. The carbon-based material 115 may also serve the function of providing a tortuous path for pathogens to traverse before the user of the air filtration system 220 is exposed to the pathogens exiting the air outlet 224.

In embodiments utilizing carbon veil as the carbon-based material 115, the carbon veil 140 includes chopped carbon fibers which are consolidated in the form of a non-woven fabric. This non-woven fabric may be held together by binder compounds, including but not limited to polymers such as acrylonitrile styrene acrylate. The heated area generated by the carbon veil varies depending on the air filtration system 220. In some embodiments, the heated area is equal to the area of a cross-section of the air flow path 226. The carbon veil 140 has a thickness that can vary between different embodiments. In some further embodiments, the carbon veil 140 has a thickness greater than or equal to 0.1 micron and less than or equal to 3 mm. In some embodiments, the carbon veil 140 has a thickness greater than or equal to 0.05 mm and less than or equal to 0.127 mm.

In embodiments utilizing the at least one CNT sheet 160 as the carbon-based material 115, the CNT sheets 160 include spinnable CNT arrays that are drawn into aligned sheets. Carbon-based heaters 116 using CNT sheets 160 exhibit ultrafast thermal response over 1000° C./s, low operation voltage, long lifetime, and lighter weight than metallic heaters. In some embodiments, a plurality of CNT sheets 160 may be arranged in cross-ply multi-layers by orienting the adjacent CNT sheets 160 perpendicularly relative to each other. The total number of CNT sheets used in the carbon-based material 115 may vary. In some embodiments, the carbon-based material 115 may include greater than or equal to 10 CNT sheets and less than or equal to 100 CNT sheets. In other embodiments, the carbon-based material 115 may include greater than or equal to 1 CNT sheet and less than or equal to 1,000 CNT sheets. The total heated area of the carbon-based heater 116 including at least one CNT sheet 160 varies depending on the air filtration system 220. In some embodiments, the total heated area is equal to the area of a cross-section of the air flow path 220.

Applying a voltage to the carbon-based heater 116 generates Ohmic heating, adding an additional protective layer to the air filtration system 220 in the form of a heat shield that provides a thermal barrier for airborne pathogens and enables their inactivation. In order to inactivate a pathogen, the temperature of the carbon-based heater 116 may be raised to a pathogen inactivation threshold temperature. This pathogen inactivation threshold temperature may depend on the pathogen to be inactivated. For some pathogens, including SARS-CoV-2, the pathogen inactivation temperature is at least 65° C. Other pathogens that may be inactivated using heat include, by way of example and not limitation, coronaviruses, tuberculosis bacteria (Mycobacterium tuberculosis), human immunodeficiency virus (HIV), Ebola virus, biological weapons, and other organic airborne contaminants. The voltage needed to raise the temperature of the carbon-based heater 116 to the pathogen inactivation threshold depends on the size of the carbon-based heater 116 used. Generally speaking, the larger the area of the carbon-based heater 116, the larger voltage is required to reach the pathogen inactivation threshold temperature. In some embodiments, the pathogen inactivation threshold temperature can be reached by applying a voltage greater than or equal to 0.3 V and less than or equal to 50 V. In some further embodiments, the pathogen inactivation threshold temperature can be reached by applying a voltage greater than or equal to 3 V and less than or equal to 17 V. In some even further embodiments, the pathogen inactivation threshold temperature can be reached by applying a voltage greater than or equal to 3 V and less than or equal to 9 V.

### Example 1

Pieces of personal protective equipment in accordance with principles of the invention were prepared and tested. In particular, pieces of personal protective equipment including an ASTM level 3 facemask, carbon veil heater, and a nylon insulating layer were tested to determine the degree of heat insulation offered by different nylon insulating layers. The various pieces of personal protective equipment were tested by heating the pieces of personal protective equipment via the carbon veil heater and orienting an infrared camera at the inner mask layer of the pieces of personal protective equipment.

Materials

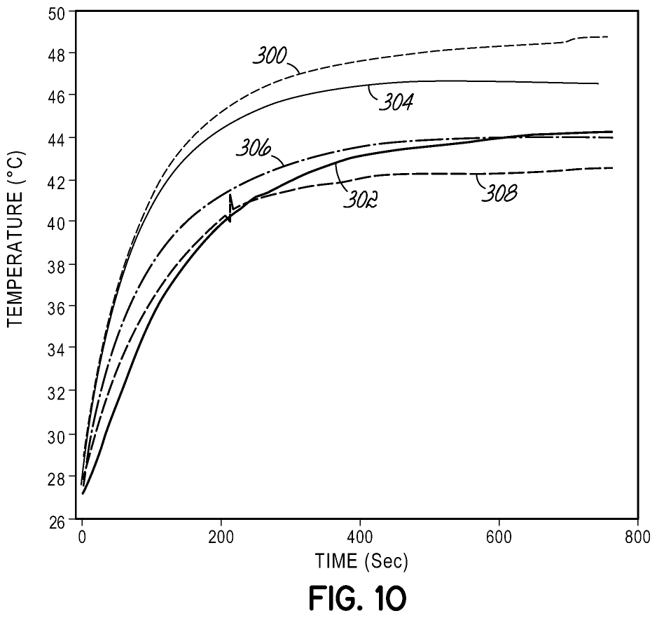

Across the various test samples, a carbon veil with a thickness greater than or equal to 10 microns and less than or equal to 200 microns was used with a preferred thickness of 127 microns. As shown in FIG. 10, the various nylon insulating layers included (A) a 1.2 mm thick insulating layer with square holes having a length of 2 mm; (B) a 2.1 mm thick insulating layer with square holes having a length of 2 mm; (C) a 1.1 mm thick insulating layer with square holes having a length of 5 mm; (D) a 2.0 mm thick insulating layer with square holes having a length of 5 mm; and (E) a 2.9 mm thick insulating layer with square holes having a length of 5 mm.

A curve 300 is obtained when one nylon insulator including square holes having a side length of 2 mm and a thickness of 1.2 mm is used. A curve 302 is obtained when one nylon insulator including square holes having a side length of 2 mm and a thickness of 2.1 mm is used. The 304 curve is obtained when one nylon insulator including square holes having a side length of 5 mm and a thickness of 1.1 mm is used. A curve 306 is obtained when one nylon insulator including square holes having a side length of 5 mm and a thickness of 2.0 mm is used. A curve 308 is obtained when one nylon insulator including square holes having a side length of 5 mm and a thickness of 2.9 mm is used.

Discussion

When comparing the various nylon insulating layers including square holes having a length of 2 mm, the data shows that increasing the thickness of the insulating layer decreased the rate of temperature increase and the final temperature measurement. When comparing the various nylon insulating layers including square holes having a length of 5 mm, the data also shows that increasing the thickness of the insulating layer decreased the rate of temperature increase and the final temperature measurement. Further, when comparing nylon insulators having approximately 1.1 mm thickness but with square holes having different side lengths, square holes with larger lengths demonstrated decreased rates of temperature increase and lower final temperature measurements. The difference in rates of temperature increase and final temperature measurements were less pronounced between nylon heaters having approximately 2.0 mm thickness having square holes having different side lengths as the thickness of the nylon insulating layers increased. In fact, the final temperature of the 2 mm side length squares was actually slightly higher than the final temperature of the 5 mm side length squares. However, as the 5 mm side length square nylon insulator thickness was increased to 2.9, rates of temperature increase and final temperature measurements were further decreased.

### Example 2

Pieces of personal protective equipment in accordance with principles of the invention were prepared and tested. In particular, pieces of personal protective equipment including an ASTM level 3 facemask, carbon veil heater, and a nylon insulating layer and pieces of personal protective equipment including a carbon veil heater and a PLA insulating layer were tested to determine the degree of heat insulation offered by different insulating layers. The various pieces of personal protective equipment were tested by heating the pieces of personal protective equipment via the carbon veil heater and orienting an infrared camera at the inner mask layer of the pieces of personal protective equipment.

Materials

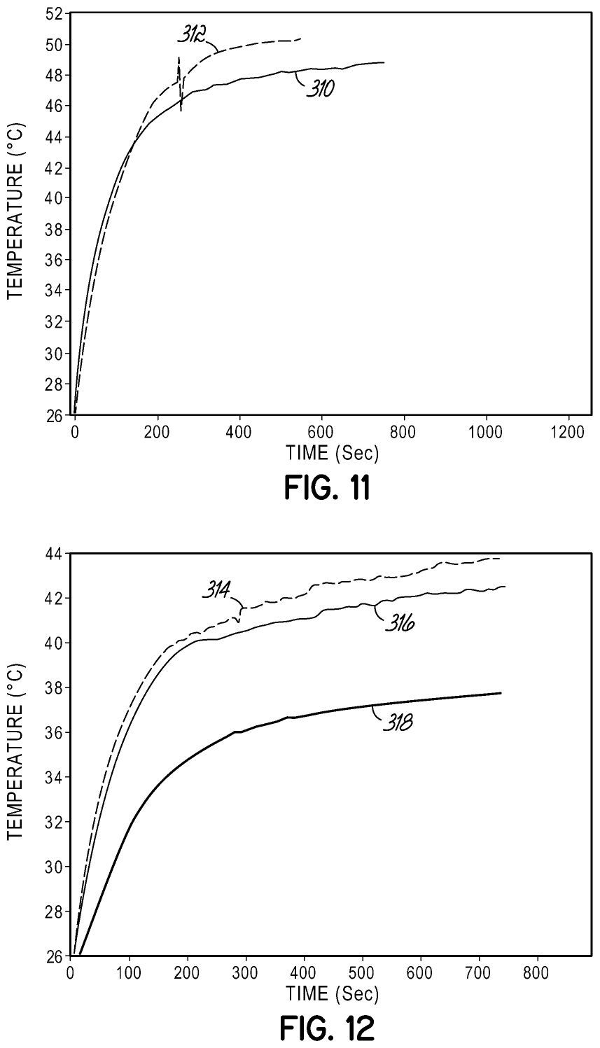

Across the various test samples, a carbon veil with a thickness greater than or equal to 10 microns and less than or equal to 200 microns was used with a preferred thickness of 127 microns. As shown in FIG. 11, one piece of personal protective equipment included a nylon insulating layer having a thickness of 1.2 mm with square holes having a length of 2 mm. The other piece of personal protective equipment included a PLA insulating layer having a thickness of 0.8 mm with square holes having a length of 3 mm.

A curve 310 is obtained when one nylon insulator including square holes having a side length of 2 mm and a thickness of 1.2 mm is used. A curve 312 is obtained when one PLA insulator including square holes having a side length of 3 mm and a thickness of 0.8 mm is used.

Discussion

When comparing the nylon insulating layer to the PLA insulating layer, the data shows that nylon insulating layer decreased the rate of temperature increase and decreased the final temperature measurement.

### Example 3

Pieces of personal protective equipment in accordance with principles of the invention were prepared and tested. In particular, pieces of personal protective equipment including an ASTM level 3 facemask, carbon veil heater, and at least one polyester membrane insulating layer were tested to determine the degree of heat insulation offered by different polyester insulating layers. In some embodiments, the polyester membrane insulating membranes were perforated. The various pieces of personal protective equipment were tested by heating the pieces of personal protective equipment via the carbon veil heater and orienting an infrared camera at the inner mask layer of the pieces of personal protective equipment.

Materials

Across the various test samples, a carbon veil with a thickness greater than or equal to 10 microns and less than or equal to 200 microns was used with a preferred thickness of 127 microns. As shown in FIG. 12, a first piece of personal protective equipment included a polyester membrane insulating layer having a thickness of 3.5 mm. A second piece of personal protective equipment included a perforated polyester membrane insulating layer having a thickness of 3.5 mm and circular holes having a diameter of 3.5 mm. A third piece of personal protective equipment included two perforated polyester membrane insulating layers each having a thickness of 3.5 mm and circular holes having a diameter of 3.5 mm.

A curve 314 is obtained when one non-perforated polyester membrane insulator having a thickness of 3.5 mm is used. A curve 316 is obtained when one perforated polyester insulator including circular holes having a diameter of 3 mm and a thickness of 3.5 mm is used. A curve 318 is obtained when two perforated polyester insulators including circular holes having a diameter of 3 mm and each having a thickness of 3.5 mm is used.

Discussion

When comparing the results of a polyester membrane to a perforated polyester membrane, the perforation resulted in a decreased rate of temperature increase and a decreased final temperature measurement. Further, when comparing the results of the perforated polyester membrane to the two layered perforated polyester membrane, there was a larger decrease in both the rate of temperature increase and the final temperature measurement.

### Example 4

Pieces of personal protective equipment in accordance with principles of the invention were prepared and tested. In particular, pieces of personal protective equipment including a carbon veil heater and a nylon insulating layer were tested to determine the pressure drop caused by different nylon insulating layers. The various pieces of personal protective equipment were tested by cutting a coupon of each layer and measuring the difference between a controlled air flow using a manometer attached to both sides of the piece of personal protective equipment.

Materials

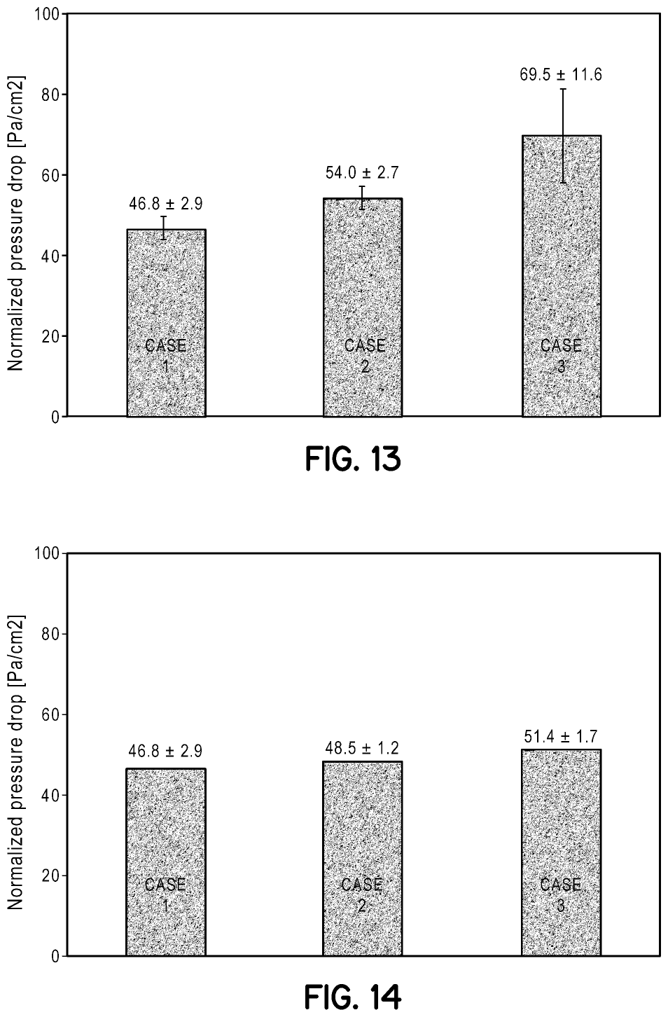

Across the various test samples, a carbon veil with a 127 microns thickness and an ASTM level 3 commercial facemask were used. As shown in FIG. 13, the various pieces of personal protective equipment tested included nylon insulating layers having square holes with 2 mm side lengths of varying thicknesses. The tested pieces of personal protective equipment included (1) no nylon insulating layer; (2) a 1.2 mm thick nylon insulating layer; and (3) a 2.1 mm thick nylon insulating layer.

Discussion

When comparing the various nylon insulating layers including square holes having a length of 2 mm, the data shows that increasing the thickness of the insulating layer increased the pressure drop through the heatable mask. As shown in the first sample without the insulating layer, the ASTM level 3 facemask together with the carbon veil heater exhibited a pressure drop of 46.8±2.9 Pa/cm², significantly lower than the ASTM International Standard F2100-19 requirement that the pressure drop be less than or equal to 58.8 Pa/cm². The inclusion of a nylon insulating layer having a thickness of 1.2 mm resulted in a pressure drop of 54.0±2.7 Pa/cm², still below the ASTM International Standard F2100-19 pressure drop requirement. However, the inclusion of a nylon insulating layer having a thickness of 2.1 mm resulted in a pressure drop of 69.5±11.6 Pa/cm², which exceeded the ASTM International Standard F2100-19 pressure drop requirement.

### Example 5

Pieces of personal protective equipment in accordance with principles of the invention were prepared and tested. In particular, pieces of personal protective equipment including a carbon veil heater and a nylon insulating layer were tested to determine the pressure drop caused by different nylon insulating layers. The various pieces of personal protective equipment were tested by cutting a coupon of each layer and measuring the difference between a controlled air flow using a manometer attached to both sides of the piece of personal protective equipment.

Materials

Across the various test samples, a carbon veil with a 127 microns thickness and an ASTM level 3 commercial facemask were used. As shown in FIG. 14, the various pieces of personal protective equipment tested included nylon insulating layers having square holes with 5 mm side lengths of varying thicknesses. The tested pieces of personal protective equipment included (1) no nylon insulating layer; (2) a 2 mm thick nylon insulating layer; and (3) a 2.9 mm thick nylon insulating layer.

Discussion

When comparing the various nylon insulating layers including square holes having a length of 5 mm, the data shows that increasing the thickness of the insulating layer increased the pressure drop through the piece of personal protective equipment. As shown in the first sample without the insulating layer, the ASTM level 3 facemask together with the carbon veil heater exhibited a pressure drop of 46.8±2.9 Pa/cm², significantly lower than the ASTM International Standard F2100-19 requirement that the pressure drop be less than or equal to 58.8 Pa/cm². The inclusion of a nylon insulating layer having a thickness of 2 mm resulted in a pressure drop of 48.5±1.2 Pa/cm², still below the ASTM International Standard F2100-19 pressure drop requirement. The inclusion of a nylon insulating layer having a thickness of 2.9 mm resulted in a pressure drop of 51.4±1.7 Pa/cm², which was also below the ASTM International Standard F2100-19 pressure drop requirement.

When compared to the nylon insulating barriers having square holes with side lengths of 2 mm, nylon insulating barriers having square holes with side lengths of 5 mm exhibit lower pressure drop increases at similar thicknesses. For example, while a 2 mm square hole with a thickness of 2.1 mm exceeded the ASTM International Standard F2100-19 pressure drop requirement, the 5 mm square hole with a thickness of 2 mm did not exceed the pressure drop requirement.

While the present invention has been illustrated by the description of one or more embodiments thereof, and while the embodiments have been described in considerable detail, they are not intended to restrict or in any way limit the scope of the appended claims to such detail. The various features shown and described herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of Applicants' general inventive concept.

What is claimed is:

1. An apparatus comprising:
a piece of personal protective equipment; and
a carbon-based material configured to trap a pathogen.

2. The apparatus of claim 1, wherein the piece of personal protective equipment further comprises a filter.

3. The apparatus of claim 2, wherein the piece of personal protective equipment is a mask, and wherein the mask comprises:
an inner layer; and
an outer layer;
wherein the carbon-based material is disposed relative to the mask at a location selected from the group consisting of an external surface of the inner layer, an external surface of the outer layer, and between the inner and outer layers.

4. The apparatus of claim 3, wherein the carbon-based material is disposed adjacent to the external surface of the outer layer.

5. The apparatus of claim 3 further comprising:
a plurality of electrodes;
a plurality of wires;
a power source; and
a switch;
wherein the plurality of electrodes, plurality of wires, power source, and switch are operatively connected to the carbon-based material, to form a carbon-based heater.

6. The apparatus of claim 5, wherein the carbon-based heater can reach a pathogen inactivation threshold temperature when a voltage is applied to the carbon-based heater.

7. The apparatus of claim 5, wherein the carbon-based material comprises a carbon veil.

8. The apparatus of claim 6, wherein the carbon veil has a thickness greater than or equal to 0.05 mm and less than or equal to 3.0 mm.

9. The apparatus of claim 5, wherein the carbon-based material comprises a carbon nanotube sheet.

10. The apparatus of claim 9, wherein the carbon-based material comprises a plurality of carbon nanotube sheets.

11. The apparatus of claim 10, wherein the plurality of carbon nanotube sheets are oriented perpendicularly relative to each adjacent carbon nanotube sheet.

12. The apparatus of claim 11, wherein the plurality of carbon nanotube sheets are perforated.

13. The apparatus of claim 5, wherein the carbon-based heater is configured to be powered on while the piece of personal protective equipment is not worn by a user, and wherein the piece of personal protective equipment is configured to be reused after heat is applied to the piece of personal protective equipment.

14. The apparatus of claim 5 further comprising an insulating layer.

15. The apparatus of claim 14, wherein the insulating layer is configured to be positioned between the carbon-based heater and the face of a user.

16. The apparatus of claim 15, wherein the insulating layer is positioned between the carbon-based heater and the outer layer.

17. The apparatus of claim 14, wherein the insulating layer has a thickness greater than or equal to 0.1 mm and less than or equal to 3 mm, and wherein the insulating layer comprises woven fiberglass fabric.

18. The apparatus of claim 14, wherein the insulating layer has a thickness greater than or equal to 1 mm and less than or equal to 6 mm, and wherein the insulating layer comprises a material selected from the group consisting of a polyester membrane, a silica aerogel, an alumina aerogel, polylactic acid, polyhydroxyalkanoate, cellulose, polyester, starch, polyvinyl alcohol, natural sill, and natural wood.

19. The apparatus of claim 14, wherein the insulating layer has a thickness greater than or equal to 0.1 mm and less than or equal to 5 mm, and wherein the insulating layer comprises a material selected from the group consisting of nylon, polylactic acid, and a fiberglass screen.

20. The apparatus of claim 14, wherein the insulating layer comprises holes having a cross-section with a shape, wherein the diameter or side length of the shape is greater than or equal to 0.1 mm and less than or equal to 10 mm.

21. The apparatus of claim 20, wherein the shape is selected from the group consisting of a circle, a semi-circle, a triangle, a square, a hexagon, and other polygons capable of being repeated over a 2D plane.

22. The apparatus of claim 14, wherein the insulating layer is made by 3D printing.

23. The apparatus of claim 14, wherein the carbon-based heater and the insulating layer are attached to the piece of personal protective equipment using a method of binding components together selected from a group consisting of conventional sewing, ultrasonic sewing, Velcro, and hot pressing in the presence of a polymer adhesive.

24. The apparatus of claim 5, wherein the power source is a battery.

25. The apparatus of claim 5, wherein the power source is a connector configured to receive power from an external power source.

26. The apparatus of claim 5, wherein the carbon-based heater is configured to be powered on for at least sixty seconds to eradicate all pathogens trapped on the piece of personal protective equipment.

27. The apparatus of claim 5, wherein the carbon-based heater is configured to be removable from and reattachable to the piece of personal protective equipment.

28. An air filtration system comprising:
an air flow path comprising:
an air inlet;
an air flow controller;
a carbon-based material configured to trap a pathogen; and
an air outlet;
wherein the carbon-based material is positioned between the air inlet and the air outlet.

29. The air filtration system of claim 28 further comprising a filter positioned between the air inlet and the air outlet.

30. The air filtration system of claim 29 further comprising
a plurality of electrodes;
a plurality of wires;
a power source; and
a switch;
wherein the plurality of electrodes, plurality of wires, power source, and switch are operatively connected to the carbon-based material, to form a carbon-based heater.

31. The air filtration system of claim 30, wherein the carbon-based heater can reach a pathogen inactivation threshold temperature when a voltage is applied to the carbon-based heater.

32. The air filtration system of claim 30, wherein the carbon-based material comprises carbon veil.

33. The air filtration system of claim 30, wherein the carbon-based material comprises a perforated carbon nanotube sheet.
